(19) United States
(12) Patent Application Publication
(10) Pub. No.: US 2008/0262401 A1
(43) Pub. Date: Oct. 23, 2008

(54) ORTHOSIS/ORTHESES

(75) Inventors: Helmut Wagner, Duderstadt (DE); Klaus Lidolt, Duderstadt (DE); Matthias Vollbrecht, Herzberg (DE)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Otto Bock Healthcare IP GmbH & Co. KG, Duderstadt (DE)

(21) Appl. No.: 12/108,232

(22) Filed: Apr. 23, 2008

(30) Foreign Application Priority Data
Apr. 23, 2007 (DE) 10 2007 019526.7

(51) Int. Cl. A61F 5/00

(52) U.S. Cl. 602/19; 602/36

(57) ABSTRACT

An orthesis with a base plate designed to fit the body of an orthesis wearer enables improved handling and storage. The base plate being provided on at least one side with a fastening mechanism for rigid connection with a separate extension piece for enlarging the base plate and the extension piece to be connected to the base plate. The extension piece also provided with a similar fastening mechanism, such that multiple extension pieces may be connected together.

### CROSS-REFERENCE TO RELATED APPLICATION

This patent application claims priority from German Patent Application No. 10 2007 019526.7, filed on Apr. 23, 2007, entitled “ORTHOSIS/ORTHESES”, the entire contents of which are hereby expressly incorporated by reference.

### TECHNICAL FIELD

The invention relates to an orthesis with a dimensionally stable base plate designed to fit the body of an orthesis wearer.

### BACKGROUND

Ortheses having bas plates are known in a variety of configurations. The base plate can be any plate which fits flat on the body of the orthesis wearer by way of appropriate fastening means. For example, a ventral base plate is located in the waist region, with a fastening strap encircling the body in this waist region. A dorsal back board can also be provided, which is looped round by the fastening strap or which has fastening mechanisms for the corresponding ends of the fastening strap, so that the fastening strap encircles the body together with the back board.

A principal field of application of such an orthesis is a hyperextension orthesis, in which at least another support board projects away from the base plate in the direction of height, therefore upwards and/or downwards, attached stably to the base plate. For a complete hyperextension orthesis an upper sternal board and a lower symphitic board cooperate ventrally with the back board to form a triangular support. A function of the base plate is to act as a fastening element for the fastening strap and both ventral boards. This type of hyperextension orthesis is known for example from DE 39 28 628 C1. Such ortheses are available in a number of sizes to enable them to fit body contours and provide a comfortable fit for the orthesis using base plates of various sizes. If a base plate is too narrow the fastening strap will cut too much into the tissue of the patient, while a base plate which is too wide will result in an unsteady fit. In the case of known ortheses, the base plate and the boards include a metal part and a padded lining. To make up different sizes of the orthesis base plates of various sizes must therefore be kept in stock.

### SUMMARY

An of the present invention is to design an orthesis such that it enables improved handling and storage. In one embodiment, an orthesis of the of the present invention includes a base plate being on at least one side with a fastening mechanism for dimensionally stable connection with an extension piece enlarging the base plate.

Depending on whether there are no extension pieces or one, two or three of them, etc. arranged on the base plate, fitting the base plate to the body contour, or to the respectively required function of the orthesis, can be carried out without base plates of different sizes being required for each separate size. If the base plate, for example in the direction of width, has ends of a length-adjustable fastening strap encircling a body on both sides, then by widening the base plate the extension pieces help it fit on the respective body contour. The connection made according to the invention between the base plate and the fitted extension pieces is likewise dimensionally stable, and thus sets the required dimensional stability against the tractive force of the fastening strap to prevent the fastening strap from undesirably cutting into the tissue of the patient.

To form several different sizes of the resulting base plate, it is effective if the extension piece is likewise provided at its free edge with a fastening mechanism. The fastening mechanism provides a positive connection to another dimensionally stable extension piece to enlarge the base plate. In this way the abovementioned multiple stepped enlargements can be completed.

In another embodiment of the invention, the fastening mechanism has an attachment with a stepped recess and the set extension piece grips under the stepped recess with a corresponding stepped recess to form a partially flat contact between attachment and extension piece. Thus, in the direction of attachment, a stop of the extension piece rests on a stop of the attachment. This produces a highly dimensionally stable connection, made structurally rigid by the flat contact of the extension piece on the attachment of the base plate. The rigidity of the connection does not oppose the connection between base plate and extension piece and/or between extension pieces for fitting the body shape of the orthesis wearer. Rather, the pieces are designed to tilt about a limited angle and exhibit a certain elasticity, so that they can be tightened on the body for example by the tractive force of a fastening strap, but they also provide a sufficiently large resistance against deformation so as not to press too much into the tissue of the orthesis wearer.

When the extension piece is in the assembled state, a locking pin arranged perpendicularly in the region of the flat contact preferably engages in a corresponding recess to form the positive connection in the direction of width. For example, the locking pin can be guided into the recess by a tilting motion of the extension piece relative to the attachment, resulting in the locking pin being snap-locked in the recess. This produces a stable connection which can be made by simply setting and snap-locking the extension piece on the corresponding attachment of the base plate or a previous extension piece.

To form the orthesis, the base plate may include at least one calotte set in the direction of height. Use of the inventive orthesis as hyperextension orthesis with a symphitic board and a sternal board is preferred. Both the sternal board and the symphitic board are preferably arranged height-adjustably on the base plate. For this purpose one rail is preferably formed from two tubes, attached to a U-shaped end in the material of the board. Height adjustment is done by shifting the tubes with the board and by corresponding suitable fastening means of the base plate.

The present invention can be utilized with a variety of support plates to enlarge or reduce the size of the base plate by the addition or removal of extension pieces, and in particular the back board of the hyperextension orthesis. Base plates can be widened by extension pieces in a preferred exemplary embodiment. But they can also be designed in different heights, in that extension pieces, which exhibit the width of the back board, are positioned to increase the length of the back board in the direction of height and thus produce a spinal support of varying length.

The inventive orthesis preferably has a base plate, which is designed fully as an injection-moulded plastic part. The extension pieces are accordingly also solid injection-moulded plastic parts, preferably without any metal reinforcement.

### BRIEF DESCRIPTION OF THE DRAWINGS

The invention will now be explained in greater detail by way of an exemplary embodiment illustrated in the diagram, in which:

FIG. 1 is a schematic illustration of a first exemplary embodiment of an inventive orthesis designed as hyperextension orthesis;

FIG. 2 is a perspective partial illustration of a base plate and an unmounted extension piece;

FIG. 3 is the illustration according to FIG. 2 with the extension piece tilted relative to the base part for assembly;

FIG. 4 is a perspective view of the proximal faces of the parts according to FIG. 3;

FIG. 5 shows the parts according to FIG. 3 during the assembly procedure;

FIG. 6 shows the parts according to FIG. 5 in the assembled state;

FIG. 7 is a schematic illustration of a second exemplary embodiment of an orthesis designed according to the invention as hyperextension orthesis;

FIG. 8 is a schematic illustration of a third exemplary embodiment of an orthesis designed according to the invention as a hyperextension orthesis.

### DETAILED DESCRIPTION

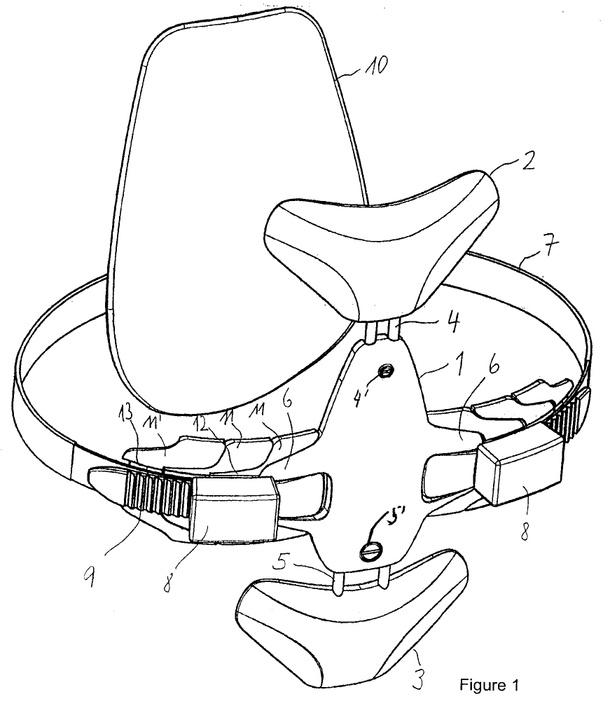

The perspective illustration of FIG. 1 of a hyperextension orthesis shows a ventral base plate 1, to which a sternal board 2 is attached upwards and a symphitic board 3 is attached downwards in the direction of height. The height position of the sternal board 2 can be adjusted, in that the sternal board 2 is attached to a rail array formed by two tubes 4 and can be shifted relative to the base plate 1. The base plate 1 has a set screw 4′ for fixing the tubes 4 connected to the sternal board 2 at a preferred height. The symphitic board 3 likewise is correspondingly height-adjustably fixed on a rail array, formed by two parallel tubes 5 fixable with a set screw 5′. The tubes 4, 5 in each case both running parallel to one another emerge as legs of a tube bent into a U-shape, whereby the U-shaped end of the arrangement is fixed in the relevant board 2, 3.

The base plate 1 has two attachments 6 pointing away from one another in the direction of width. Fixed to these attachments 6 are drive belts 9. A fastening strap 7 provided to loop around the body of a patient in the waist region has at its end a firmly mounted fastener 8. The fastener 8 can be pushed to different positions on the drive belt 9 as the patient requires, so as to thus create various peripheral tensions.

The orthesis comprises a back board 10 by which the fastening strap 7 can be pressed against the back of the patient in the chest and lumbar vertebrae region.

To adapt the orthesis to different waist girths, an extension piece 11 connects to the attachment 6 of the base plate 1, which is configured with an attachment 12 corresponding to the attachment 6, so that a further extension piece 11 etc. can be set onto the extension piece 11. In the exemplary embodiment illustrated in FIG. 1, three extension pieces 11 are set on both sides of the base plate 1 in each case, which, as explained in greater detail hereinbelow, are connected to one another positively and provide a dimensionally stable widening of the base plate 1. The last extension piece 11 is designed as an end piece 11′, which is designed on the distal surface with a grooved guide 13 for guiding the fastening strap 7.

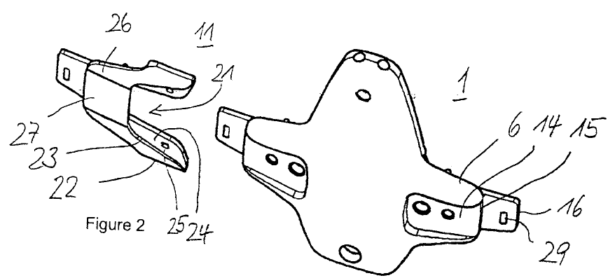

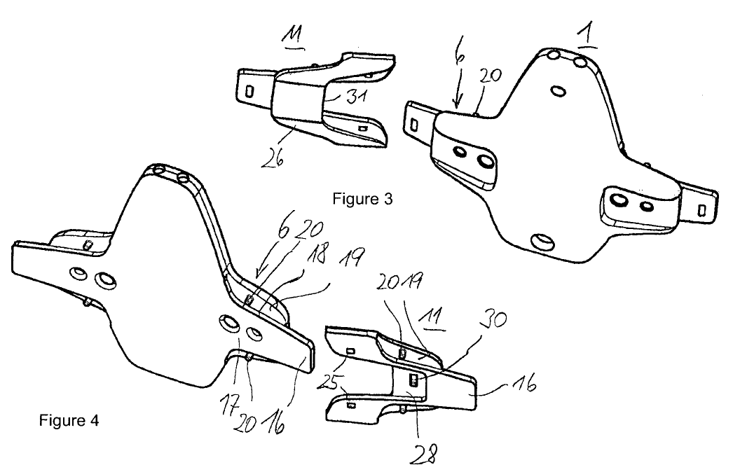

FIGS. 2 to 6 illustrate the structural design of the base plate 1 and the (same) extension pieces 11. FIGS. 2 and 3 show a perspective view of the distal sides of the base plate 1 and an extension piece 11. On the attachments 6, the base plate 1 has outwardly open grooved depressions 14, in which the ends of the fastening strap 7 are fastened by means of an associated lock 8. The attachments 6 taper conically outwards in width and terminate in an edge 15 forming a stop. Underneath, thus proximally from the edge 15, a tongue 16 extends out over the edge 15 in the direction of width.

FIG. 4 shows that on the proximal side, a tongue 16 continues a section 17 of the attachment 6. On the proximal side, the attachment section 17 extends gradually from the material of the attachment 6, such that in the direction of height on both sides of the section 17 it comprises a stepped recess 18, by which a contact surface 19 is formed set back and stepped on both sides of the section 17 relative to the surface of the section 17. A locking pin 20 extends from the contact surface 19 in each case.

The extension piece 11 has a forked end, whereof a recess 21 is designed with a contour corresponding to the outer contour of the tongue 16. The recess 21 is delimited by two webs 22, which have on their distal surface a stepped transition line 23, whereof the progression corresponds to, and thus complements, the progression of the stepped recess 18 of the attachment 6. Through the stepped transition 23 each web 22 has a stepped set-back contact surface 24, which is designed to contact the corresponding contact surface 19. The contact surface 24 is provided with an opening 25 for receiving the locking pin 20. The webs 22 are connected to one another on the far side of the recess 21 by a base body 26, which has on its distal top side a grooved recess 27 in the direction of width and which serves as guide for the fastening strap 7.

As FIG. 4 shows, the base body 26 has on its proximal side a central stepped recess 28, serving to receive the tongue 16 of the attachment 6 protruding out over the stop edge 15. The tongue 16 is provided with a snap-in cam 29 which engages in a corresponding depression 30 in the stepped recess 28 in the assembled state.

FIG. 4 also shows that, as with the attachment 6, the base body 26 of the extension piece 11 is provided correspondingly with a fastening mechanism of contact surface 19 and locking pin 20 as well as with a tongue 16. Thus, the extension body 11 can be snapped in identically with a further extension body 11.

FIG. 3 shows that locking of the extension body 11 on the attachment 6 of the base plate 1 takes place by the extension piece 11 being tilted proximally and in this position being moved to the attachment 6 such that the contact surface 24 lies opposite the contact surface 19. In the process, the stop edge 15 of the attachment 6 lies on a corresponding stop edge 31 of the base body 26. When the extension piece 11 is tilted back into the position flush with the attachment 6, the locking pins 20 engage in the openings 25 of the extension piece 11. At the same time, this results in engagement of the snap-in cam 29 of the tongue 16 in the depression 30 of the stepped recess 28 of the extension piece 11. Both locking versions can be designed to snap-lock, creating a connection positive in the direction of width both via the locking pins 20 and via the snap-in cam 29. Through corresponding dimensioning, for example of the snap-in cam 29 and depression 30, the connection can be tilted and/or configured elastically about an axis perpendicular to the direction of width about a limited angle, producing an automatic fit on the body contour of the orthesis wearer. In the direction of traction of the fastening strap 7, there is also a dimensionally stable connection made based on the nesting contact surfaces 19, 24 on both sides (in the direction of height) of the attachment 6.

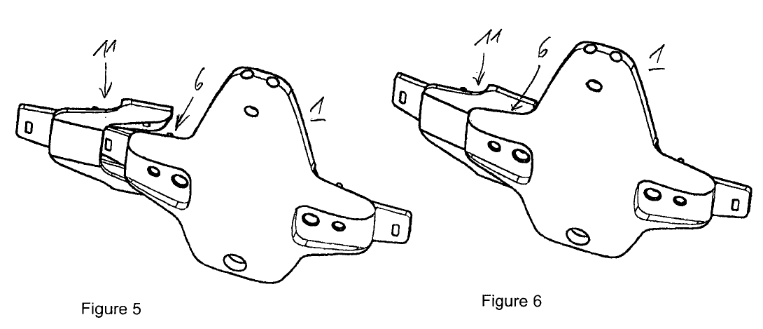

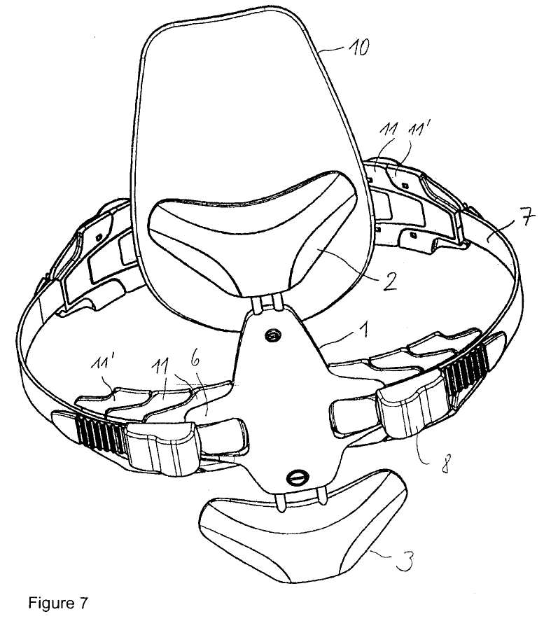

FIG. 5 shows the extension piece 11 nearing the assembly position. FIG. 6 shows the extension piece 11 set positively flush and dimensionally stable after being tilted back with the attachment 6 of the base plate 1.

FIG. 6 also shows that another identically designed extension piece similarly can be connected to the extension piece 11. Thus, the configuration illustrated in FIG. 1 can be readily created with a base part 1 widened by three extension pieces 11.

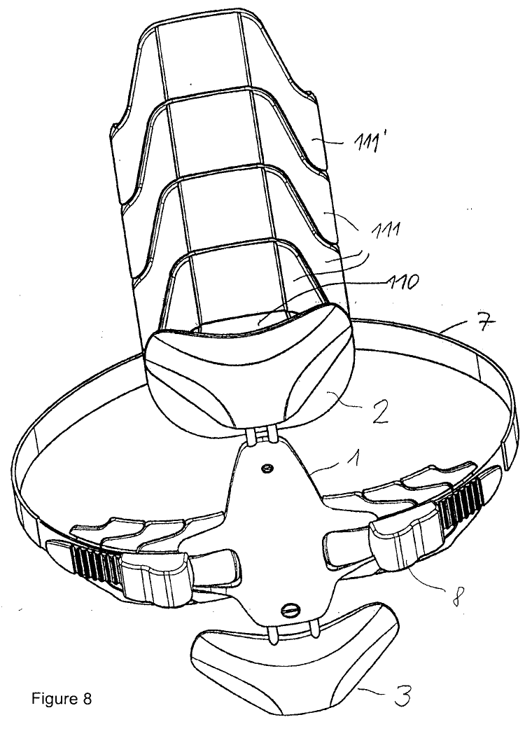

FIG. 7 shows a second exemplary embodiment of an inventive orthesis, configured as a hyperextension orthesis and differing from the first exemplary embodiment, shown in FIG. 1. In this embodiment, the back board 10 has a fastening mechanism for affixing attachment pieces 11, so that also the width of the back board 10 can be similarly adjusted, as can the width of the ventral base plate 1.

Compared to the first exemplary embodiment of FIG. 1, the third exemplary embodiment illustrated in FIG. 8 contains the modification, whereby the back board 110 is designed as base plate which can be enlarged in the direction of height. For this, extension pieces 111, extending over the entire width of the back board 110, can be connected to the back board 110. In this way, the respective length of a spinal support can be set by the back board 110. In this embodiment, it is particularly significant that the extension pieces 111 are designed to tilt relative to the base part of the back board 110 and relative to one another about a limited angle. Thus, the fit can be made to the contour of the spine, and yet the required support is ensured. As with the widening of the sides, the extension made by means of at least one extension piece 111 is closed by an end piece 111′.

It is also conceivable, of course, to combine the width-adjustable back board 10, as shown in FIG. 7, with the back board 110 that can be enlarged in the direction of height. Thus, the back board 110 can be enlarged by extension pieces 11, 111 in the direction of both width and height.

The following is claimed:

1. An orthesis designed to fit the body of an orthesis wearer, comprising:
a base plate configured for attachment to the body of the orthesis wearer;
a fastening mechanism provided on at least one side of the baseplate; and
at least one dimensionally stable extension piece configured to be coupled to the base plate at the fastening mechanism, thereby enlarging the base plate.

2. The orthesis according to claim 1, further comprising a length-adjustable fastening strap for encircling the body, the fastening strap including ends that are arranged on the base plate on both sides, and wherein the enlarged base plate provides deformation resistance against the tractive force of the fastening strap.

3. The orthesis according to claim 1, further comprising a fastening mechanism provided at a free edge of the at least one extension piece for coupling to another extension piece to further enlarge the base plate.

4. The orthesis according to claim 3, wherein the extension pieces each comprise a guide on a distal surface for the fastening strap.

5. The orthesis according to claim 1, wherein the fastening mechanism further comprises an attachment with a stepped recess and a stop, and wherein the extension piece further comprises a stop and a corresponding stepped recess that grips under the fastening mechanism stepped recess to form a partially flat contact between the attachment and extension piece, such that the extension piece rests on the stop of the attachment.

6. The orthesis according to claim 5, wherein the attachment further comprises a locking pin arranged perpendicularly in the region of the flat contact, and the extension piece further comprises a corresponding recess such that the locking pin engages the corresponding recess to form a positive connection when the extension piece is coupled to the base plate.

7. The orthesis according to claim 6, wherein the locking pin is guidable into the recess by the extension piece being tilted relative to the attachment as the extension piece is coupled to the base plate.

8. The orthesis according to claim 6, wherein the locking pin is configured to snap into the recess.

9. The orthesis according to any one of claim 1, wherein the base plate further comprises at least one board connected to the base plate in the direction of height.

10. The orthesis according to claim 9, wherein the at least one board comprises a symphitic board configured to shift downwards on the base plate in the direction of height and a sternal board configured to shift upwards in the direction of height, and wherein the orthesis further comprises a length-adjustable fastening strap for encircling the body, the fastening strap including ends that are connected to the base plate on both sides, and a back board arranged on the fastening strap, such that the orthesis is a hyperextension orthesis.

11. The orthesis according to claim 9, wherein the base plate further comprises a rail including two tubes connected to a U-shaped end embedded in the material of the base plate, and wherein the at least one board is attached height-adjustably on the rail.

12. The orthesis according to claim 1, wherein the base plate and the at least one extension piece comprise injection-moulded plastic parts.

13. The orthesis according to claim 1, wherein the connection between base plate and extension piece, and/or between one extension piece and another extension piece, is configured to tilt about a limited angle to fit the body shape of the orthesis wearer.

14. The orthesis according to claim 1, wherein the base plate and/or the at least one extension piece are elastic providing a desired fit to the body contour of the wearer, such that they provide a deformation resistance protecting the body against a tractive force.

15. The orthesis according to claim 1, further comprising a back board coupled to the base plate, the back board configured as an enlargeable base plate.

16. The orthesis according to claim 15, wherein the back board is enlargable in the direction of width by the attachment pieces.

17. The orthesis according to claim 15, wherein the back board is enlargable in the direction of height by the attachment pieces.

18. The orthesis according to claim 15, wherein the enlargeable back board is connected via a fastening strap to the enlargeable base plate.

19. A hyperextension orthesis designed to fit the body of an orthesis wearer, comprising:
a base plate configured for attachment to the body of the orthesis wearer, the base plate including a symphitic board configured to shift downwards on the base plate in the direction of height and a sternal board configured to shift upwards in the direction of height;
a fastening mechanism provided on at least one side of the baseplate; and
at least one dimensionally stable extension piece couplable to the base plate at the fastening mechanism, thereby enlarging the base plate.

20. The hyperextension orthesis of claim 19, wherein the orthesis further comprises a length-adjustable fastening strap for encircling the body, the fastening strap including ends that are connected to the base plate on both sides, and a back board arranged on the fastening strap.

21. The hyperextension orthesis of claim 20, wherein the base plate further comprises two rails, each including two tubes connected to a U-shaped end embedded in the material of the base plate, one extending downware and one extending upward from the baseplate, and wherein the symphitic and sternal boards are attached height-adjustably on the rails, respectively.
